(19) United States
(12) Patent Application Publication
(10) Pub. No.: US 2020/0197320 A1
(43) Pub. Date: Jun. 25, 2020

(54) DRUG CARRIER AND DRUG DELIVERY SYSTEM THEREOF

(71) Applicant: NATIONAL TSING HUA UNIVERSITY, HSINCHU (TW)

(72) Inventors: Chi-How Peng, Hsinchu (TW); Hung-Hsun Lu, Hsinchu (TW)

(73) Assignee: NATIONAL TSING HUA UNIVERSITY

(21) Appl. No.: 16/549,268

(22) Filed: Aug. 23, 2019

(30) Foreign Application Priority Data
Dec. 20, 2018 (TW) 107146227

(51) Int. Cl. A61K 9/51; C12N 15/113; A61K 31/704; A61K 41/00

(52) U.S. Cl. CPC A61K 9/5146; C12N 15/113; C12N 2320/32; A61K 41/0042; C12N 2310/14; A61K 31/704

(57) ABSTRACT

A drug carrier includes a structure represented by formula (1) defined as in the specification is provided. The drug carrier can be used as a drug delivery system. The drug delivery system can include the drug carrier and an effective amount of a nucleic acid, in which the nucleic acid is encapsulated in the drug carrier. The drug delivery system also can include the drug carrier and an effective amount of an active substance, in which the active substance is encapsulated in the drug carrier.

### RELATED APPLICATIONS

This application claims priority to Taiwan Application Serial Number 107146227, filed Dec. 20, 2018, which is herein incorporated by reference.

### BACKGROUND

### Technical Field

The present disclosure relates to a drug carrier and a drug delivery system thereof. More particularly, the present disclosure relates to a poly(ethylene glycol)-based block copolymer as a drug carrier and a drug delivery system thereof.

### Description of Related Art

In recent years, pharmaceutical dosage forms and formulation studies have entered the era of drug delivery systems (DDS), which use physical or chemical methods to alter the structure of the formulation so that the drug is maintained at a specific release rate in accordance with the dosage form design for a predetermined period of time, releasing the drug in a particular organ and tissue, and allowing the drug to remain in the effective concentration range for a longer period of time in the body. That is, the ideal drug delivery system enables the drug to selectively release the drug encapsulated therein after entering the human body under specific conditions.

At present, the common methods of drug delivery are oral administration and intravenous injection. Although oral administration is convenient, the pH value of the digestive tract is easy to cause damage to the drug. When the drug is injected into the blood circulation, the concentration of the drug easily exceeds the toxic concentration and causes side effects. The advantages of injection administration are that the drug is absorbed quickly, the concentration of the drug in the plasma rapidly increases, and the amount of the drug in the body is accurate. However, the injection administration can cause pain in the tissue of the injection site and easily lead to adverse reactions. That is, the above two drug delivery methods have their own bottlenecks. For clinical controlling the condition of the patient, it is necessary to increase the frequency of oral administration or injection administration to maintain the concentration of the drug in the body, which in turn causes discomfort and inconvenience to the patient.

In addition, physiologically active nucleic acid drugs such as small interfering RNA (siRNA) and antisense RNA (asRNA) are expected to be the next generation therapeutic drugs for gene therapy, anticancer or viral diseases. However, the use of the nucleic acid drugs is limited to their instability in vivo and low use efficiency in vivo. In order to apply nucleic acid drugs to the treatment of a wider range of diseases, drug delivery systems for the efficient and safe administration of nucleic acids for systemic administration are also necessary in the future and development. Viral vectors are known to deliver nucleic acids to target sites with high efficiency, but are limited in their clinical utility due to their immunogenicity and carcinogenicity. Therefore, other drug delivery systems currently under development are mainly non-viral vectors composed of cationic polymers or cationic lipids. Even though cationic polymers can stabilize nucleic acid and form a complex via electrostatic interaction, but still limited to the stability in blood.

Therefore, development of a novel drug delivery system, which can effectively deliver the nucleic acid or the active substances into the body, is crucial in the field of pharmacy.

### SUMMARY

According to one aspect of the present disclosure, a drug carrier is provided. The drug carrier includes a structure represented by formula (1):

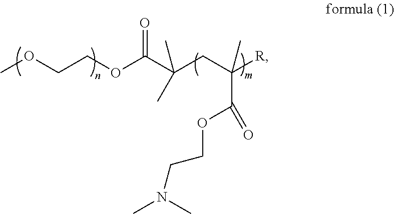

wherein n is a number greater than 30 and less than 150, m is a number greater than 30 and less than 120, R is a structure represented by formula (2) or formula (3):

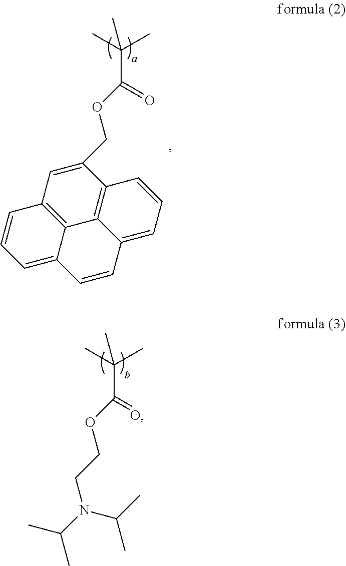

wherein a is a number greater than 30 and less than 100, b is a number greater than 30 and less than 100.

According to another aspect of the present disclosure, a drug delivery system is provided. The drug delivery system includes the drug carrier according to the aforementioned aspect and an effective amount of a nucleic acid, wherein the nucleic acid is encapsulated in the drug carrier.

According to still another aspect of the present disclosure, a drug delivery system is provided. The drug delivery system includes the drug carrier according to the aforementioned aspect and an effective amount of an active substance, wherein the active substance is encapsulated in the drug carrier.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIGS. 1A, 1B, and 1C show analytical results of characterization of a drug carrier according to one embodiment of the present disclosure.

FIGS. 2A and 2B show analytical results of cleavage of the drug carrier upon a photo irradiation according to one embodiment of the present disclosure.

FIG. 3A shows an analytical result of condensation efficiency of siRNA of a drug delivery system according to the 1st embodiment of the present disclosure.

FIG. 3B shows an analytical result of stability of a siRNA encapsulated drug delivery system according to the 1st embodiment of the present disclosure.

FIG. 4 shows an analytical result of the cumulative siRNA release of a drug delivery system of Example 1.

FIGS. 5A, 5B, 5C and 5D show analytical results of tri-phase transition of PEG₁₁₃-b-PDMAEMA₃₁-b-PMAA₃₀ carrier.

FIG. 6A shows an analytical result of the effect of a UV irradiation on cytotoxicity.

FIG. 6B shows an analytical result of cytotoxicity analysis of the drug delivery system of Example 1.

FIGS. 7A and 7B show analytical results of in vitro cellular uptake of the drug delivery system of Example 1.

FIG. 8 shows an analytical result of knockdown efficiency of the drug delivery system of Example 1.

FIGS. 9A, 9B, 9C, 9D, 9E, 9F, 9G, 9H, 9I, 9J, 9K and 9L show analytical results of characterization of a drug carrier according to another embodiment of the present disclosure.

FIG. 10 shows an analytical result of drug loading efficiency of a drug delivery system according to the 2nd embodiment of the present disclosure.

FIG. 11 is a schematic view of an active substance encapsulated in the drug delivery system according to the 2nd embodiment of the present disclosure.

FIG. 12 shows an analytical result of cumulative active substance release of a drug delivery system of Example 6.

### DETAILED DESCRIPTION

A drug carrier is provided in the present disclosure. The drug carrier includes a structure represented by formula (1):

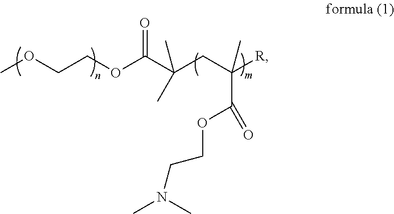

wherein n is a number greater than 30 and less than 150, m is a number greater than 30 and less than 120, R is a structure represented by formula (2) or formula (3):

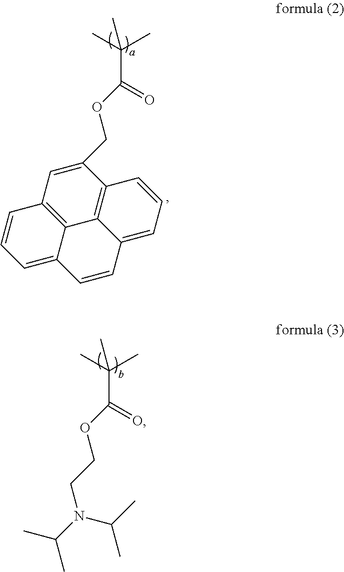

wherein a is a number greater than 30 and less than 100, b is a number greater than 30 and less than 100.

The drug carrier of the present disclosure has a cationic charge group, which can form an ionic complex with a hydrophobic compound, a nucleic acid or a hydrophobic drug. The ionic complex can self-assemble in an aqueous solution to form a micelleplex with a core-shell configuration as a drug delivery system. The micelleplex has a hydrophilic polymer chain as a shell, and a hydrophobic polymer as a core, and a nucleic acid or a hydrophobic drug is encapsulated in the core of the drug carrier.

Therefore, a drug delivery system is provided in the present disclosure. The drug delivery system includes the aforementioned drug carrier and an effective amount of a nucleic acid, wherein the nucleic acid is encapsulated in the drug carrier. Particularly, a ratio of a concentration of amine groups in the drug carrier to a concentration of phosphate groups in the nucleic acid is an N/P ratio, and the N/P ratio is higher than or equal to 5.

The nucleic acid refers to an oligonucleotide or a polynucleotide having a nucleotide consisting of a purine and/or pyrimidine, a pentose sugar, and a phosphoric acid as a basic unit. The nucleic acid can be selected from the group consisting of an oligo-double-stranded DNA, a poly-double-stranded DNA, an oligo-single-stranded DNA, a poly-single-stranded DNA, an oligo-single-stranded RNA and a poly-single-stranded RNA. The nucleic acid can be an oligo- or poly-double-stranded nucleic acid containing a mixture of RNA and DNA on the same strand, or oligo- or poly-nuclear nucleic acid containing a mixture of RNA and DNA on the same strand. Furthermore, the nucleotide contained in the nucleic acid can be a natural type or a chemically modified non-natural type. The nucleotide contained in the nucleic acid can have an amine group, a thiol group or a fluorescent compound. The nucleic acid can have 4 to 100 bases, preferably 10 to 50 bases, and more preferably 18 to 30 bases. Classified by function, the nucleic acid can be a plasmid DNA, a small interfering RNA (siRNA), a microRNA (miRNA), an antisense RNA (asRNA), a decoy nucleic acid or an aptamer. The siRNA can be a single-stranded or double-stranded RNA of 18 to 30 nucleotides in length, which can be designed according to the gene to be subjected to gene therapy. The asRNA refers to an RNA molecule having a complementary sequence to a target RNA, and specifically blocks the translated RNA or DNA molecule by base pairing with the target RNA to participate in the regulation of gene expression. The nucleic acid can be released by a photo irradiation or by lowering a pH value to a release pH value, wherein the release pH value is less than or equal to 5.

Another drug delivery system is also provided in the present disclosure. The drug delivery system includes the aforementioned drug carrier and an effective amount of an effective amount of an active substance, wherein the active substance is encapsulated in the drug carrier. Particularly, a loading concentration of the active substance in the drug carrier is greater than 0 mg/mL and less than 100 mg/mL. The active substance can be a hydrophobic drug. Preferably, the hydrophobic drug can be doxorubicin (DOX), tamoxifen, irinotecan, paclitaxel or sorafenib. Further, the active substance can be released by the photo irradiation or by lowering a pH value to a release pH value, wherein the release pH value is less than or equal to 5.

The present disclosure will be further exemplified by the following specific embodiments so as to facilitate utilizing and practicing the present disclosure completely by the people skilled in the art without over-interpreting and over-experimenting. However, the readers should understand that the present disclosure should not be limited to these practical details thereof, that is, these practical details are used to describe how to implement the materials and methods of the present disclosure and are not necessary.

### EXAMPLES AND COMPARATIVE EXAMPLES

### 1st Embodiment

A poly(ethylene glycol)-based block copolymer containing an amine as a drug carrier is provided in the present disclosure, which includes a structure represented by the formula (1). In the 1st embodiment, the poly(ethylene glycol)-based block copolymer contains a photo-responsive cleavage segment. Particularly, R in the formula (1) of the drug carrier of the 1st embodiment is a structure represented by formula (2):

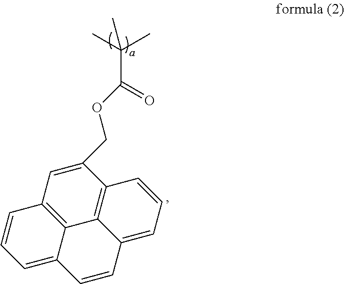

wherein a is a number greater than 30 and less than 100. More particularly, the drug carrier of the 1st embodiment has a structure represented by formula (4):

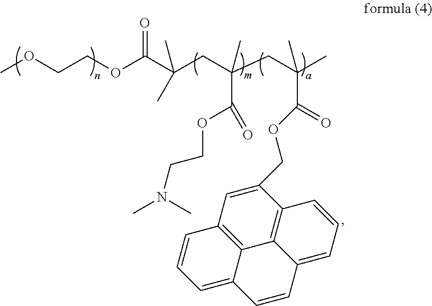

wherein n is the number greater than 30 and less than 150, m is the number greater than 30 and less than 120, a is the number greater than 30 and less than 100. That is, the drug carrier of the 1st embodiment is a triblock copolymer of PEGₙ-b-PDMAEMAₙ-b-PPyₐ obtained by polymerizing poly(ethylene glycol) (PEG), poly(2-dimethylaminoethyl methacrylate) (PDMAEMA), and poly(pyrenylmethyl methacrylate) (PPy).

### 1.1. Synthesis and Characterization of a Drug Carrier of the 1st Embodiment

PEG₁₁₃ is utilized as a reference segment of PEG to polymerize with different size PDMAEMA segments and different size PPy segments, forming the drug carrier of the 1st embodiment with different molecular weight. The drug carrier of the 1st embodiment is prepared by atom-transfer radical-polymerization (ATRP) technique, which can form the polymer evenly and easily. The drug carrier is obtained by sequentially addition of monomers to a polymerization reaction. First, the PEG₁₁₃-Br segment is prepared as a radical initiator, the monomer of 2-dimethylaminoethyl methacrylate (DMAEMA) is polymerized with the PEG₁₁₃-Br segment, and then the monomer of pyrenylmethyl methacrylate (Py) is added for undergoing chain extension to form a triblock copolymer of PEG₁₁₃-b-PDMAEMAₘ-b-PPyₐ. However, the method for preparing the drug carrier of the 1st embodiment is not limited to the aforementioned. There are three examples of the drug carrier of the 1st embodiment, which are the drug carrier of Example 1, the drug carrier of Example 2, and the drug carrier of Example 3. In addition, diblock copolymers obtained by polymerizing a PEG segment with different size PDMAEMA segments are prepared as comparative examples. There are three comparative examples, which are the drug carrier of Comparative Example 1, the drug carrier of Comparative Example 2, and the drug carrier of Comparative Example 3.

The number average molecular weight (M_{n,GPC}), the weight average molecular weight (M_{w,GPC}), and the polydispersity index (PDI, Ð) of the prepared drug carrier of Examples 1 to 3 and the drug carrier of Comparative Examples 1 to 3 are measured by gel permeation chromatography (GPC), wherein the polydispersity index (PDI) is calculated as the weight average molecular weight (M_{w,GPC}) divided by the number average molecular weight (M_{n,GPC}). The ¹H-NMR spectrum is measured by nuclear magnetic resonance (NMR) so as to confirm the molecular structure, and the copolymer composition and the number average molecular weight (M_{n,NMR}) of the drug carrier of the 1st embodiment can also be determined. The particle size distribution of the drug carrier of Examples 1 to 3 is measured by dynamic laser scattering (DLS), and the particle size of the drug carrier of Example 1 is also measured by transmission electron microscope (TEM).

After confirmation by nuclear magnetic resonance, the repeating unit number of each segment of the drug carrier of Example 1 is PEG₁₁₃-b-PDMAEMA₃₁-b-PPy₃₀, the repeating unit number of each segment of the drug carrier of Example 2 is PEG₁₁₃-b-PDMAEMA₇₀-b-PPy₂₈, the repeating unit number of each segment of the drug carrier of Example 3 is PEG₁₁₃-b-PDMAEMA₁₀₅-b-PPy₃₂, the repeating unit number of each segment of the drug carrier of Comparative Example 1 is PEG₁₁₃-b-PDMAEMA₃₁, the repeating unit number of each segment of the drug carrier of Comparative Example 2 is PEG₁₁₃-b-PDMAEMA₈₀, and the repeating unit number of each segment of the drug carrier of Comparative Example 3 is PEG₁₁₃-b-PDMAEMA₁₀₅.

Further, the analytical results of the number average molecular weight (M_{n,NMR}), the polydispersity index (PDI) and the particle size distribution of the drug carrier of Example 1, the drug carrier of Example 2, the drug carrier of Example 3, the drug carrier of Comparative Example 1, the drug carrier of Comparative Example 2 and the drug carrier of Comparative Example 3 are shown in Table 1.

TABLE 1

| Drug carrier | M_{n, NMR} (g mol⁻¹) | PDI | Size (nm) |
|---|---|---|---|
| PEG₁₁₃—Br | 5,000 | 1.03 | — |
| Example 1 | 18,900 | 1.38 | 83 |
| Example 2 | 24,400 | 1.40 | 102 |
| Example 3 | 31,100 | 1.33 | 118 |
| Comparative Example 1 | 9,900 | 1.27 | — |
| Comparative Example 2 | 17,700 | 1.30 | — |
| Comparative Example 3 | 21,500 | 1.31 | — |

As shown in Table 1, the polydispersity index of the drug carrier of Example 1, the drug carrier of Example 2, and the drug carrier of Example 3 of the present disclosure ranges from 1.1 to 2.0, wherein the PDI value is closer to 1, the molecular weight of the polymer is more uniform. The results indicate that the molecular weight of the drug carrier of PEGₙ-PDMAEMAₘ-PPyₐ of the 1st embodiment is distributed uniformly.

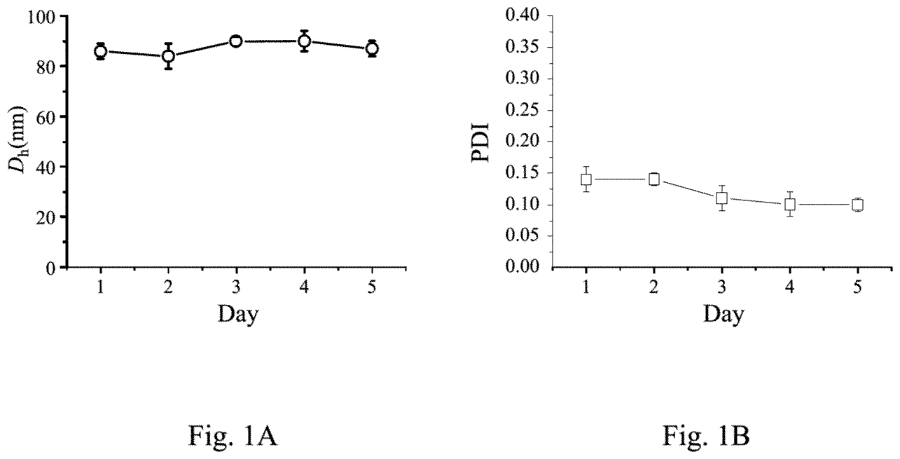

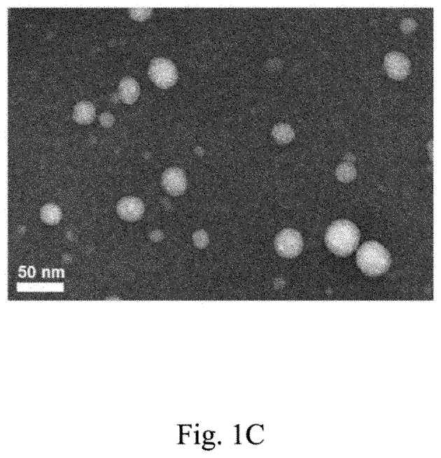

Please refer to FIGS. 1A, 1B and 1C. FIGS. 1A and 1B show the analytical results of dynamic laser scattering analysis of the drug carrier of Example 1, wherein FIG. 1A shows the analytical result of time-dependent hydrodynamic diameter (Dₕ) of the drug carrier of Example 1, and FIG. 1B shows the analytical result of time-dependent particle dispersity (PDI) of the drug carrier of Example 1. FIG. 1C is a transmission electron microscope (TEM) image of the drug carrier of Example 1 for confirming the particle size of the drug carrier of Example 1.

In FIGS. 1A and 1B, 50 μg/mL of the drug carrier of Example 1 can be self-assembled by typical nanoprecipitation under sonication. The light-intensity-average hydrodynamic diameter the drug carrier of Example 1 is 83 nm with uniform dispersity of 0.11, and the drug carrier of Example 1 retains homogeneous dispersity (PDI<0.14) with size around 85 nm in five days at 25° C. The high colloidal stability of the drug carrier of Example 1 is attributed to the π-π stacking interaction between pyrene molecules inside the hydrophobic core. In FIG. 1C, the drug carrier of Example 1 is negatively stained by phosphotungstic acid (PTA), which presents a spherical shape with white core of approximately 40 nm in diameter, indicating the formation of core-corona micelles with a hydrophobic core formed by PPy segment stacking and hydrophilic outer spherical surface composed by PEG segment and PDMAEMA segment. The 7 different particle sizes of the drug carrier of Example 1 observed by DLS analysis and TEM images could be resulted from shrinkage of PEG segment and PDMAEMA segment when water is removed. This is because DLS analysis is performed in water solution, whereas the TEM sample is pre-treated by the air-dried method.

### 1.2. Stimuli-Responsiveness of the Drug Carder of the 1st Embodiment

The drug carrier of the 1st embodiment is expected to be responsive to the photo irradiation because the drug carrier of the 1st embodiment contains the photo-responsive cleavage segment. The drug carrier of Example 1 is selected as a model drug carrier of the 1st embodiment tested in this experiment. UV-light of 365 nm in wavelength can cleave the ester bond on the PPy segment of the drug carrier of Example 1, thus converting poly(pyrenylmethyl methacrylate) to poly(methacrylic acid) (PMAA). Accordingly, the photo-responsiveness of the drug carrier of Example 1 is first characterized by the electronic absorption and emission spectrum.

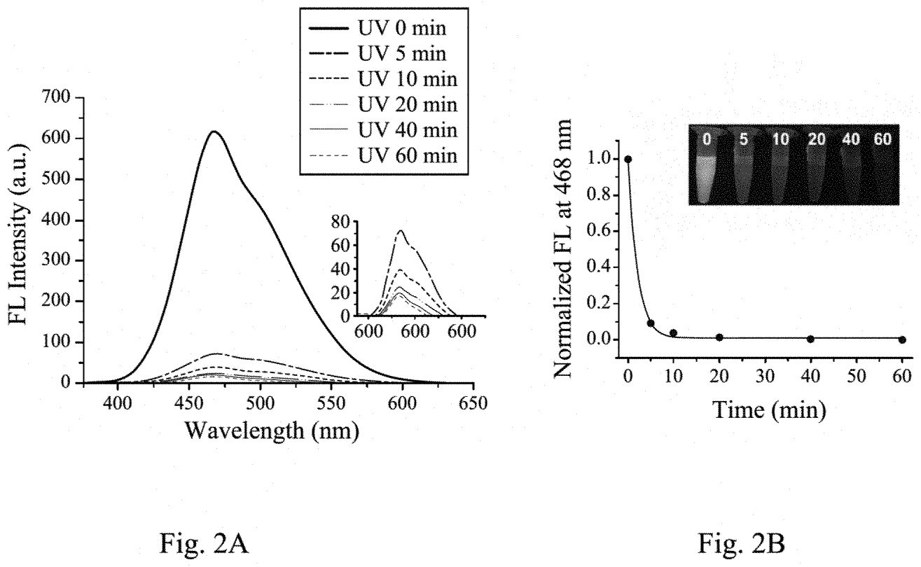

Please refer to FIGS. 2A and 2B, which show analytical results of cleavage of the drug carrier upon the photo irradiation according to one embodiment of the present disclosure. FIG. 2A shows time-dependent fluorescence spectra of the drug carrier of Example 1. FIG. 2B is a normalized time-dependent fluorescent intensity at 468 nm of the drug carrier of Example 1. In FIGS. 2A and 2B, characteristic absorption bands of PPy segment is identified at wavelength above 300 nm by using the UV-vis spectroscopy.

In FIGS. 2A and 2B, the fluorescence spectrum with excitation at 365 nm illustrate the typical peak for excimers at 468 nm with the range from 400 to 600 nm, which is attributed to the stacking of pyrene molecules in a constrained space. The fluorescent intensity of the drug carrier of Example 1 at 468 nm decreased as the UV irradiation time increased and dropped rapidly to 4% of the original intensity within 10 minutes, after which it stayed relatively constant. Additionally, the water insolubility of detached pyrene derivatives, mainly pyrenemethanol, also contributes to the drastic reduction of excimer fluorescence as well as the absence of monomeric pyrene emission. As a result, cleavage of photolabile ester bond on PPy segment is considered efficient by the photo irradiation. In addition, the photo-responsiveness of the drug carrier of Example 1 caused by the cleavage of PPy ester bond affects not only the fluorescent intensity but also the nanostructure. The particle size of the drug carrier of Example 1 in water is decreased from 88 nm to 65 nm in diameter within 10 minutes under the UV irradiation and subsequently reaching 60 nm in 30 minutes. The significant changes in fluorescent intensity and particle size both occur within 10 minutes under UV irradiation, suggesting that 10 minutes of UV irradiation is sufficient to convert PPy segment to PMAA segment with the hydrophobic core disassembled.

### 1.3. Condensation Efficiency and Release Analysis of SiRNA of a Drug Delivery System of the 1st Embodiment

The drug carrier of the 1st embodiment is expected to carry the siRNA efficiently via the electrostatic interaction between positively-charged amine groups on the PDMAEMA segment and negatively-charged phosphate groups on the siRNA to form a drug delivery system of the 1st embodiment. The ratio of a concentration of amine groups in the drug carrier to a concentration of phosphate groups in the nucleic acid is an N/P ratio, which is used to assess the condensation efficiency of the drug delivery system of the present disclosure.

The solutions with different concentration of the drug carrier of Example 1, the drug carrier of Example 2, and the drug carrier of Example 3 are added into siRNA solution at stock concentration to formulate the drug delivery system of Example 1, the drug delivery system of Example 2, and the drug delivery system of Example 3 with different N/P ratios. In addition, the drug carrier of Comparative Example 1, the drug carrier of Comparative Example 2, and the drug carrier of Comparative Example 3 that are reported to show high siRNA condensation efficiency are also used to formulate the drug delivery system of the Comparative Example 1, the drug delivery system of Comparative Example 2, and the drug delivery system of Comparative Example 3. Because the influence of the chain length of the drug delivery system of Example 1, the drug delivery system of Example 2, and the drug delivery system of Example 3 to the siRNA condensation efficiency is still ambiguous, the siRNA condensation efficiency of the drug delivery system of Examples and the drug delivery system of Comparative Examples is then evaluated by using ethidium bromide assay (EBA), respectively.

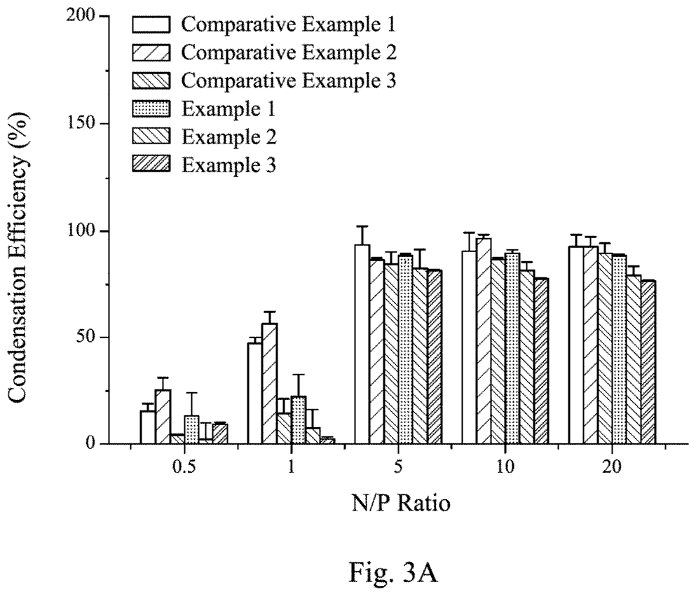

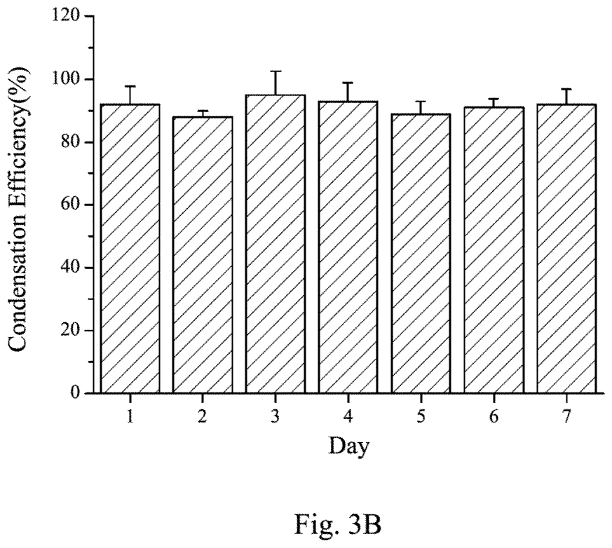

Please refer to FIG. 3A, which shows an analytical result of condensation efficiency of siRNA of a drug delivery system according to the 1st embodiment of the present disclosure. In FIG. 3A, it is clear that more siRNA is wrapped inside the drug delivery system of Example 1, the drug delivery system of Example 2, and the drug delivery system of Example 3 when the N/P ratio increased, where the efficiency reached a plateau around 90% when the N/P ratio is higher than or equal to 5. Once the N/P ratio is higher than 5, the siRNA condensation efficiency of the drug delivery system of Example 1, the drug delivery system of Example 2, the drug delivery system of Example 3, the drug delivery system of Comparative Example 1, the drug delivery system of Comparative Example 2, and the drug delivery system of Comparative Example 3 becomes quite comparable. In addition, a decrease in particle size of the drug delivery system of Example 1 from 87 nm to 72 nm is observed after the siRNA condensation and should be caused by the electrostatic interaction between PDMAEMA segment and siRNA, which contributed to the contraction of the PDMAEMA shell. Further, in order to determine the stability of the drug delivery system of the 1st embodiment after condensation the siRNA, the drug delivery system of Example 1 with the N/P ratio of 5 is experimentally stored in ultrapure water and measured its siRNA condensation efficiency every day for 7 days. Please refer to FIG. 3B, which shows an analytical result of stability of the siRNA encapsulated drug delivery system according to the 1st embodiment of the present disclosure. In FIG. 3B, the siRNA condensation efficiency of the drug delivery system of Example 1 is maintained more than 90% from day 1 to day 7, indicating that it is highly stable. As a result, the low N/P ratio, the comparable condensation efficiency of siRNA to the diblock copolymer, and the great stability demonstrated that the drug delivery system of the 1st embodiment can be an ideal drug delivery system for siRNA delivery.

The drug delivery system of Example 1 is treated with the UV irradiation for 30 minutes or without the UV irradiation in a phosphate buffer solution with pH 6.0, respectively, to evaluate the cumulative siRNA release efficiency of the drug delivery system of the 1st embodiment upon the photo irradiation.

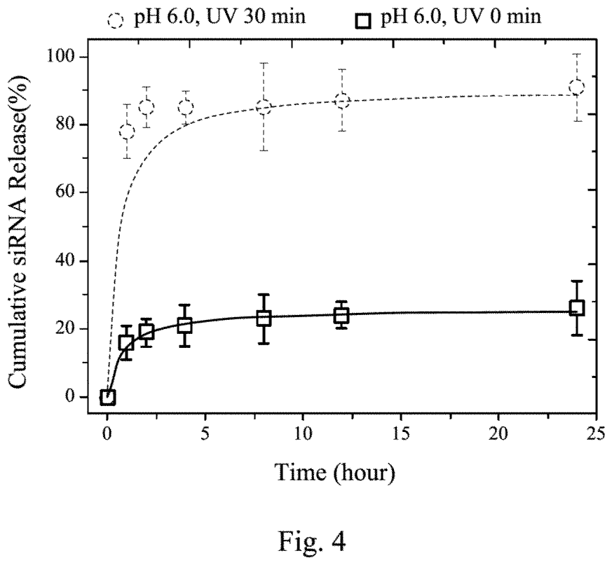

Please refer to FIG. 4, which shows an analytical result of the cumulative siRNA release of the drug delivery system of Example 1. At pH 6.0, the drug delivery system of Example 1 without treating the UV irradiation releases around 16% siRNA within one hour and then the cumulative release efficiency reaches a plateau at 26% for 24 hours. When the pH is increased to 7.4, the releasing rate approaches 34% in 24 hours (the result is not shown) because the environment of higher pH value reduces the protonation of PDMAEMA, which in turn weakens the binding between siRNA and the drug carrier of the present disclosure. Nevertheless, the drug delivery system of the 1st embodiment and siRNA still effectively minimized the uncontrolled release of siRNA and thus should possess high stability in physiological condition. By contrast, the cumulative release of siRNA of the drug delivery system of Example 1 treated with the UV irradiation for 30 minutes is 78% in one hour and further approaches 91% within 24 hours, implying that the negatively charged carboxylic groups formed from the PPy segment after photo-degradation can contribute to the release of encapsulated siRNA. The significant contrast of siRNA releasing rate before and after the UV irradiation (26% versus 91%) in FIG. 4 guarantees the drug delivery system of the present disclosure as an ideal siRNA delivery system for gene therapy.

### 1.4. Tri-Phase Transition of the Drug Delivery System of the 1st Embodiment

The coexistence of remarkably high stability and siRNA releasing rate shown by the drug delivery system of the 1st embodiment and siRNA is attributed to not only the effectively photo-triggered conversion from PPy to PMAA but also the phase transition of the drug delivery system of the 1st embodiment in different pH environment. The PEG₁₁₃-b-PDMAEMA₃₁-b-PMAA₃₀ particles produced by the photo irradiation of the drug carrier of Example 1 are stored in phosphate buffer solution with different pH values. The hydrodynamic diameter and the light-scattering intensity of the PEG₁₁₃-b-PDMAEMA₃₁-b-PMAA₃₀ particle under different conditions are analyzed by dynamic light scattering (DLS).

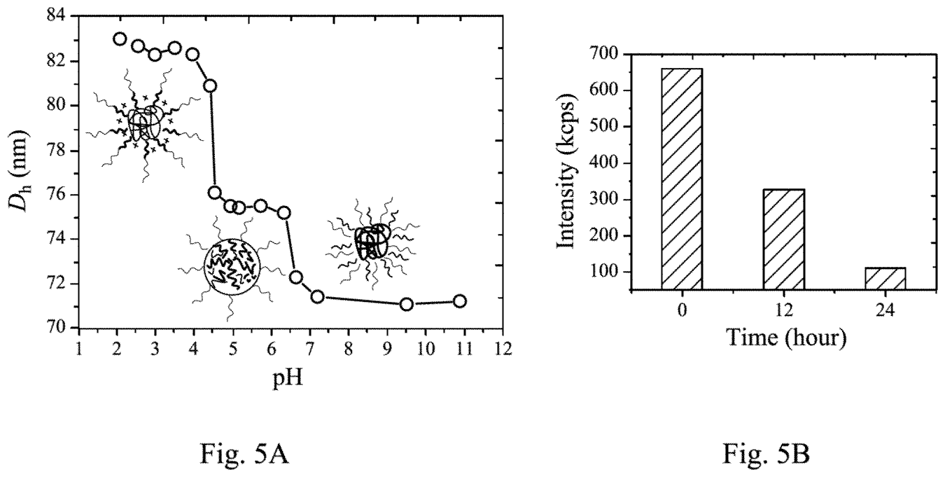

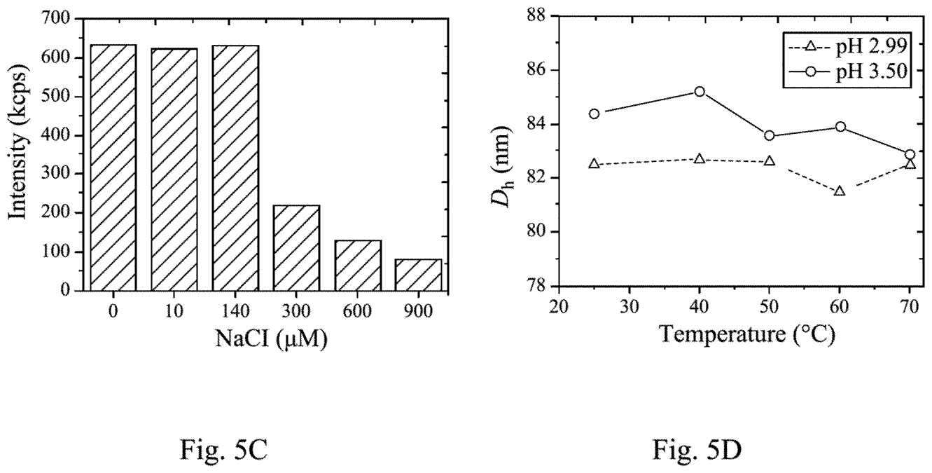

Please refer to FIGS. 5A, 5B, 5C and 5D, which show analytical results of tri-phase transition of PEG₁₁₃-b-PDMAEMA₃₁-b-PMAA₃₀ particle. FIG. 5A shows the analytical result of the hydrodynamic diameter of the PEG₁₁₃-b-PDMAEMA₃₁-b-PMAA₃₀ particle under varied pH environment. FIG. 5B shows the analytical result of the light-scattering intensity of the PEG₁₁₃-b-PDMAEMA₃₁-b-PMAA₃₀ particle at pH 8.21 with time. FIG. 5C shows the analytical result of the light-scattering intensity of the PEG₁₁₃-b-PDMAEMA₃₁-b-PMAA₃₀ particle at pH 5.8 with various ionic strengths. FIG. 5D shows the analytical result of the hydrodynamic diameter of the PEG₁₁₃-b-PDMAEMA₃₁-b-PMAA₃₀ particle at pH 2.99 and pH 3.5 with various temperatures.

In FIG. 5A, according to the pH dependent DLS measurement, three self-assembled morphologies of the PEG₁₁₃-b-PDMAEMA₃₁-b-PMAA₃₀ particle are observed. The hydrodynamic diameter of the PEG₁₁₃-b-PDMAEMA₃₁-b-PMAA₃₀ particle is 83 nm at pH 2-4, the hydrodynamic diameter of the PEG₁₁₃-b-PDMAEMA₃₁-b-PMAA₃₀ particle is 75 nm at pH 4-6, and the hydrodynamic diameter of the PEG₁₁₃-b-PDMAEMA₃₁-b-PMAA₃₀ particle is 71 nm at pH 7-11. In FIG. 5B, the decreased light intensity of the PEG₁₁₃-b-PDMAEMA₃₁-b-PMAA₃₀ particle with time in DLS measurement at pH 8.2 indicates the instability of these particles, possibly due to the weak hydrophobicity of unprotonated PDMAEMA segment.

The core of the PEG₁₁₃-b-PDMAEMA₃₁-b-PMAA₃₀ particle should be formed by positively charged PDMAEMA and negatively charged PMAA via intra-micellar electrostatic interaction to provide a consistent size of 75 nm at pH 4.3 to 6.5, which covers the range of pH 5.8-6.1 as the isoelectric point 45 estimated by the measured pKa of PDMAEMA segment and reported pKa of PMAA segment. In FIG. 5C, with the increasing ionic strength given by the addition of NaCl, the light scattering intensity shows a significant drop when the NaCl concentration reaches 300 μM. This is because the electrostatic interaction between PDMAEMA segment and PMAA segment is susceptible to the high ionic strength that causes the disassembling of particles. The characterization of particle structure rationalized the burst release of siRNA as high as 91% at pH 6.0 because the photo-triggered conversion from PPy segment to PMAA segment not only removes the hydrophobic core to destabilize the drug delivery system of the 1st embodiment but also forms the negatively charged carboxyl groups to knock off the siRNA from PDMAEMA segment by competing the positively charged amine groups with siRNA.

When the pH goes lower than 4.3, the PEG₁₁₃-b-PDMAEMA₃₁-b-PMAA₃₀ particle shows a sharp elevation in particle size up to 83 nm. The most possible structure of the PEG₁₁₃-b-PDMAEMA₃₁-b-PMAA₃₀ particle at this stage should be a PMAA segment core associated with the corona composed by PEG segment and PDMAEMA segment chains due to the hydrophobicity of undissociated PMAA segment. Although the particle core formed by PEG segment and PMAA segment via the hydrogen bonding has also been reported, the consistence in particle size of the PEG₁₁₃-b-PDMAEMA₃₁-b-PMAA₃₀ particle under varying temperature from 25° C. to 70° C. (FIG. 5D) excludes this possibility because the particles should have become unstable at high temperature if the hydrogen bond are the dominated interaction to form the micelles.

### 1.5. Material Cytotoxicity of the Drug Delivery System of the 1st Embodiment

The cytotoxicity is a crucial issue in the development of gene delivery system. For example, the systems with densely cationic charges that induce cell membrane disruption have shown the significant cytotoxicity and thus limited application. The polyethylenimine, an effective transfection vector, is hindered from in viva experiment or clinical use due to the significant cytotoxicity as well. However, the material cytotoxicity can be reduced by the micellization or pegylation that shields the cationic charges. In the drug delivery system of the 1st embodiment, both the UV irradiation and the functional groups on the drug carrier would raise the concern of cytotoxicity.

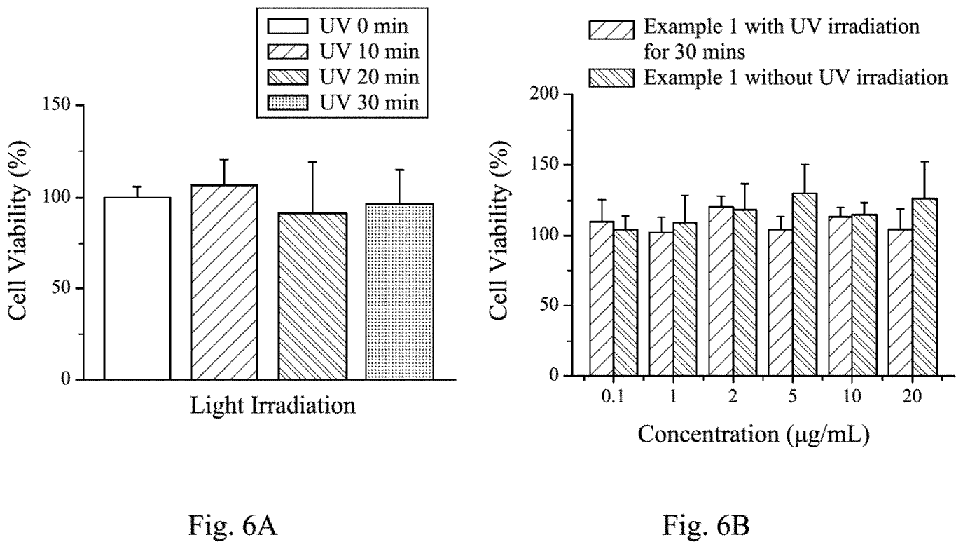

Thus, the MDA-MB-231 human breast cancer cells are exposed to the UV irradiation for 10, 20, and 30 minutes to evaluate the influence of the UV irradiation to the cell viability using MTT assay. Please refer to FIG. 6A, which shows the analytical result of the effect of the UV irradiation on cytotoxicity. Compared to the control group without the UV irradiation, the cell viability is comparable in all trials, which indicates that the cytotoxicity caused by the UV irradiation is negligible.

Then the drug delivery systems of Example 1 with different concentrations are tested for their cytotoxicity. The range of micelle concentration was selected from 0.1 μg/mL to 20 μg/mL because 20 μg/mL of the drug delivery system of Example 1 with the N/P ratio of 5 is able to condense 160 nM of siRNA, which is much higher than the effective dose of siRNA (30 nM) for activating RISC in vitro. The MDA-MB-231 human breast cancer cells are treated with 0.1, 1, 2, 5, 10 and 20 μg/mL of the drug delivery system of Example 1 for 6 hours, respectively, and then further incubated for another 24 hours. The cell viability is measured by the MTT assay. Please refer to FIG. 6B, which shows the analytical result of cytotoxicity analysis of the drug delivery system of Example 1. According to the cell viability, the drug delivery system of Example 1 of 0.1 μg/mL to 20 μg/mL does not significantly induce cytotoxicity. The results in FIGS. 6A and 6B demonstrate that no matter the drug delivery system of the 1st embodiment or the PEG₁₁₃-b-PDMAEMA₃₁-b-PMAA₃₀ particle and pyrenemethanol, the major products after the UV irradiation, are not harmful to the cells under the condition of the present disclosure.

### 1.6. In Vitro Cellular Uptake of the Drug Delivery System of the 1st Embodiment

The ability of the drug delivery system of the 1st embodiment to deliver siRNA into cells is demonstrated in this experiment. The MDA-MB-231 human breast cancer cells are co-cultured with free siRNA or the drug delivery system of Example 1 carrying the same siRNA for 4 hours, wherein the siRNA is labeled with FAM. The cellular uptake efficiency of the drug delivery system of the 1st embodiment is analyzed by confocal laser scanning microscopy (CLSM) detection and average optical density (AOD) evaluation.

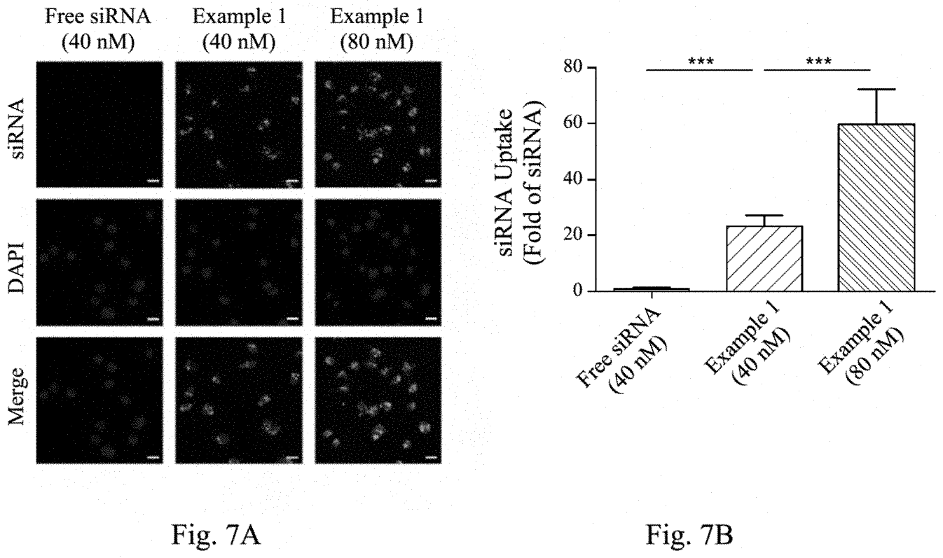

Please refer to FIGS. 7A and 7B, show the analytical results of in vitro cellular uptake of the drug delivery system of Example 1. As expected, free siRNA exhibits very low uptake efficiency because the electrostatic repulsion between negatively-charged cell membrane and siRNA could interfere cellular internalization. In contrast, the negative charges of siRNA would be shielded by the complexation with the drug carriers and thus siRNA carried by the drug delivery system of Example 1 demonstrated significantly enhanced uptake efficiency in the MDA-MB-231 human breast cancer cells, which approaches 23-fold higher than that of free siRNA. In FIG. 7B, the increasing siRNA concentration from 40 nM to 80 nM can further elevate the uptake efficiency to 60 folds, revealing a proper correlation between the number of the drug delivery system of Example 1 and the quantity of siRNA engulfed by cancer cells. The enhanced cellular internalization of siRNA carried by the drug delivery system of Example 1 should be rationalized by caveolar endocytosis that is reported as the pathway for micelleplexes to be engulfed by the cells. Notably, 5 μg/mL of the drug delivery system of Example 1, which is demonstrated to be nontoxic to the MDA-MB-231 human breast cancer cells (in Experiment 1.5), is sufficient to carry 40 nM of siRNA. Therefore, the gene knockout efficiency of the drug delivery system of the 1st embodiment is evaluated as follows at a condition of 5 μg/mL of the drug delivery system of Example 1.

### 1.7. Knockdown Efficiency of the Drug Delivery System of the 1st Embodiment

The knockdown efficiency of the drug delivery system of the 1st embodiment is demonstrated in this experiment. The MDA-MB-231 human breast cancer cells are divided into 6 groups in the experiment. Group 1 is the MDA-MB-231 human breast cancer cells without any treatment as the control group. The experimental group includes of 5 groups. Group 2 is the MDA-MB-231 human breast cancer cells treated with the UV irradiation for 30 minutes. Group 3 is the MDA-MB-231 human breast cancer cells treated with free GAPDH siRNA and the drug carrier of Example 1. Group 4 is the MDA-MB-231 human breast cancer cells treated with the drug delivery system of Example 1 encapsulated with the control siRNA, wherein the control siRNA cannot target the GADPH mRNA. Group 5 is the MDA-MB-231 human breast cancer cells treated with the drug delivery system of Example 1 encapsulated with 40 nM of GAPDH siRNA. Group 6 is the MDA-MB-231 human breast cancer cells treated with the drug delivery system of Example 1 encapsulated with 40 nM of GAPDH siRNA and then treated with the UV irradiation for 30 minutes. The silencing effect of siRNA delivered by the drug delivery system of Example 1 in the MDA-MB-231 human breast cancer cells is evaluated by measuring GAPDH enzyme activity with a KDalert GAPDH assay kit, which is assessed by the fluorescence increment with predetermined time in cell lysates. The knockdown efficiency was calculated by the ratio of remaining expression of GAPDH between the conditions with a given transfection and cell only.

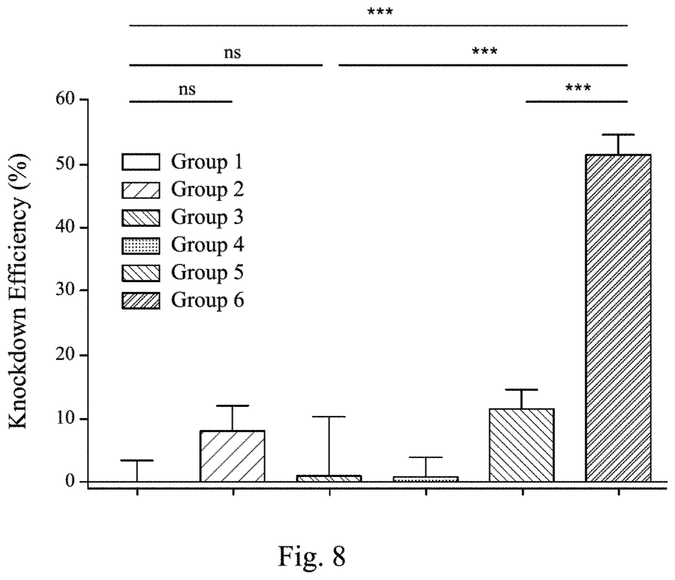

Please refer to FIG. 8, which shows the analytical result of knockdown efficiency of the drug delivery system of Example 1. Comparing to the group 1 of control group, the group 3 in which treated with the free GAPDH siRNA and the drug carrier of Example 1 and the group 4 in which treated with the drug delivery system of Example 1 encapsulated with the control siRNA show almost no knockdown efficiency because free GAPDH siRNA could not penetrate the cell membrane and the control siRNA cannot target the GADPH mRNA. Although the group 2 in which treated with the UV irradiation shows 8% knockdown efficiency in MDA-MB-231 human breast cancer cells, this value is not significantly different to that of control group from statistic aspect. The 40 nM of GAPDH siRNA delivered by the drug delivery system of Example 1 in the group 5 shows 11% knockdown efficiency possibly due to the leakage of siRNA from the drug delivery system of Example 1. However, the knockdown efficiency of the group 6 in which the drug delivery system of Example 1 with 40 nM of GAPDH siRNA and then treated with UV irradiation for 30 minutes after cellular internalization approaches 51%, indicating that the UV irradiation successfully liberate the GAPDH siRNA from the drug delivery system of Example 1 and thus activate RISC to degrade targeted GAPDH rnRNA as indicated by reduced GAPDH enzyme activity. The remarkable difference in knockdown efficiency of GAPDH siRNA encapsulated in the drug delivery system of Example 1 before the UV irradiation (group 5) and after the UV irradiation (group 6) demonstrates that the drug delivery system of Example 1 can achieve an effective gene delivery and the phototriggered gene release for cancer treatment in spatiotemporal precision.

### 2nd Embodiment

In the 2nd embodiment, the poly(ethylene glycol)-based block copolymer contains a pH-responsive cleavage segment. Particularly, R in the formula (1) of the drug carrier of the 2nd embodiment is a structure represented by formula (3):

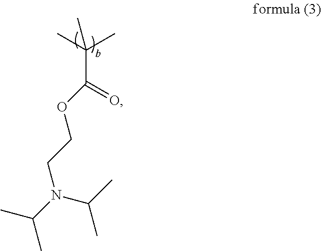

wherein b is a number greater than 30 and less than 100. More particularly, the drug carrier of the first embodiment has a structure represented by formula (5):

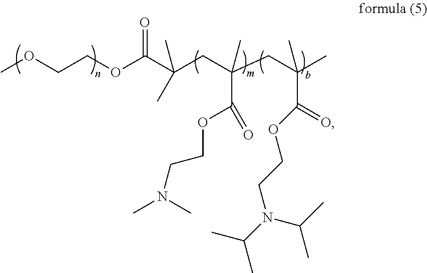

wherein n is the number greater than 30 and less than 150, m is the number greater than 30 and less than 120, b is the number greater than 30 and less than 100. That is, the drug carrier of the 2nd embodiment is a triblock copolymer of PEGₙ-b-PDMAEMAₘ-b-PDPA_{b} or a random copolymer of PEGₙ-b-(PDMAEMAₘ-r-PDPA_{b}) obtained by polymerizing poly(ethylene glycol) (PEG), poly(2-dimethylaminoethyl methacrylate) (PDMAEMA), and poly(2-(diisopropylamino)ethyl methacrylate) (PDPA).

### 2.1. Synthesis and Characterization of a Drug Carrier of the 2nd Embodiment

PEG₁₁₃ is utilized as a reference segment of PEG to polymerize with different size PDMAEMA segments and different size PDPA segments, forming the drug carrier of the 2nd embodiment with different molecular weight. The triblock copolymer of PEGₙ-b-PDMAEMAₘ-b-PDPA_{b} is prepared by atom-transfer radical-polymerization (ATRP) technique, which can form the polymer evenly and easily. The drug carrier is obtained by sequentially addition of monomers to a polymerization reaction. First, the PEG₁₁₃-Br segment is prepared as a radical initiator, the monomer of 2-dimethylaminoethyl methacrylate (DMAEMA) is polymerized with the PEG₁₁₃-Br segment, and then the monomer of 2-dimethylaminoethyl methacrylate (DPA) is added for undergoing chain extension to form a triblock copolymer of PEG₁₁₃-b-PDMAEMAₘ-b-PDPA_{b}. The random copolymer of PEGₙ-b-(PDMAEMAₘ-PDPA_{b}) is prepared by ATRP technique. First, the PEG₁₁₃-Br segment is prepared as a radical initiator, the monomer of DMAEMA the monomer of DPA are simultaneously polymerized with the PEG-₁₁₃-Br segment to form a random copolymer of PEG₁₁₃-b-(PDMAEMAₘ-r-PDPA_{b}). However, the method for preparing the drug carrier of the 2nd embodiment is not limited to the aforementioned. There are six examples of the drug carrier of the 2nd embodiment, which are the drug carrier of Example 4, the drug carrier of Example 5, the drug carrier of Example 6, the drug carrier of Example 7, the drug carrier of Example 8, and the drug carrier of Example 9.

The number average molecular weight (M_{n,GPC}), the weight average molecular weight (M_{w,GPC}), and the polydispersity index (PDI, Ð) of the prepared drug carrier of Examples 4 to 9 are measured by gel permeation chromatography (GPC), wherein the polydispersity index (PDI) is calculated as the weight average molecular weight (M_{w,GPC}) divided by the number average molecular weight (M_{n,GPC}). The ¹H-NMR spectrum is measured by nuclear magnetic resonance (NMR) so as to confirm the molecular structure, and the copolymer composition and the number average molecular weight (M_{n,NMR}) of the drug carrier of the 2nd embodiment can also be determined. The degree of polymerization (DP) of DMAEMA and DPA is also measured by nuclear magnetic resonance.

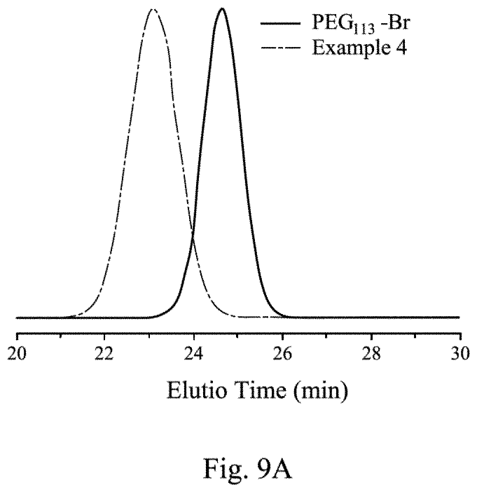

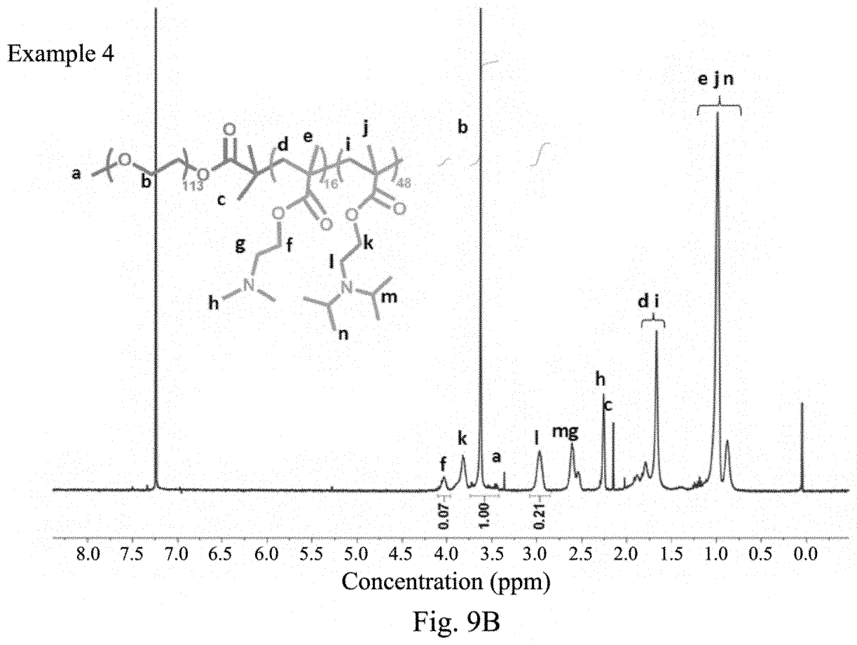

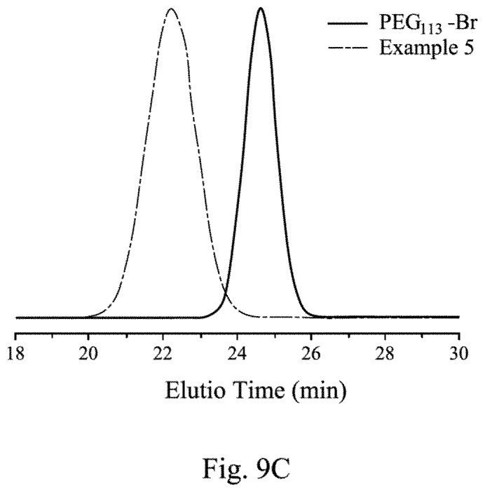

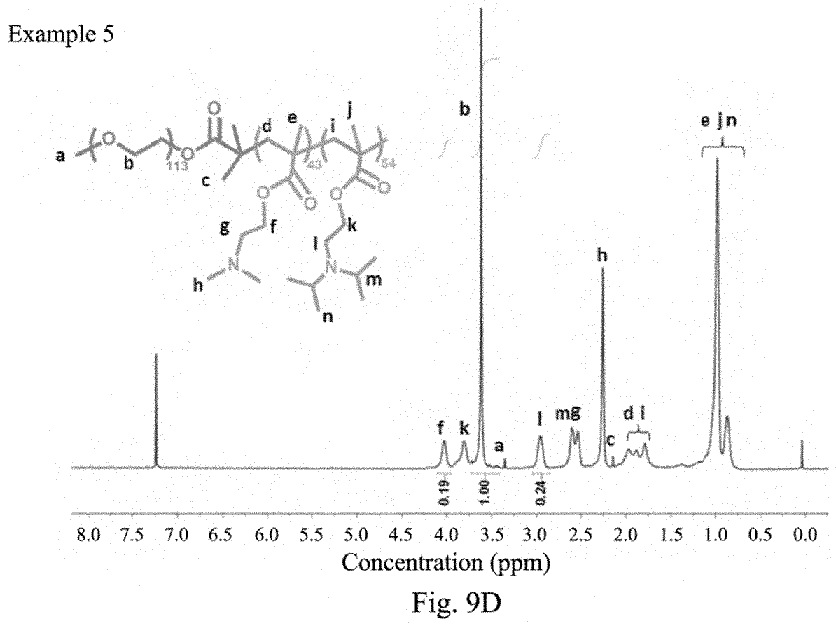

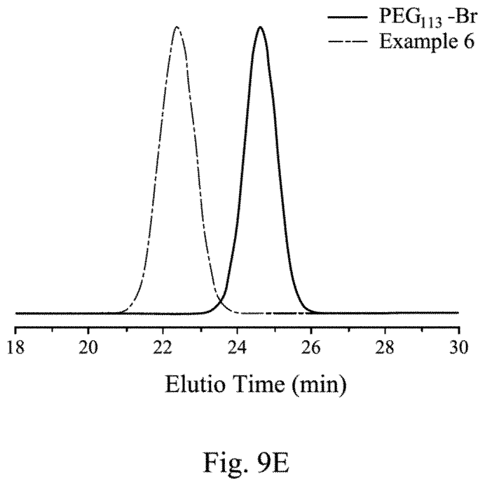

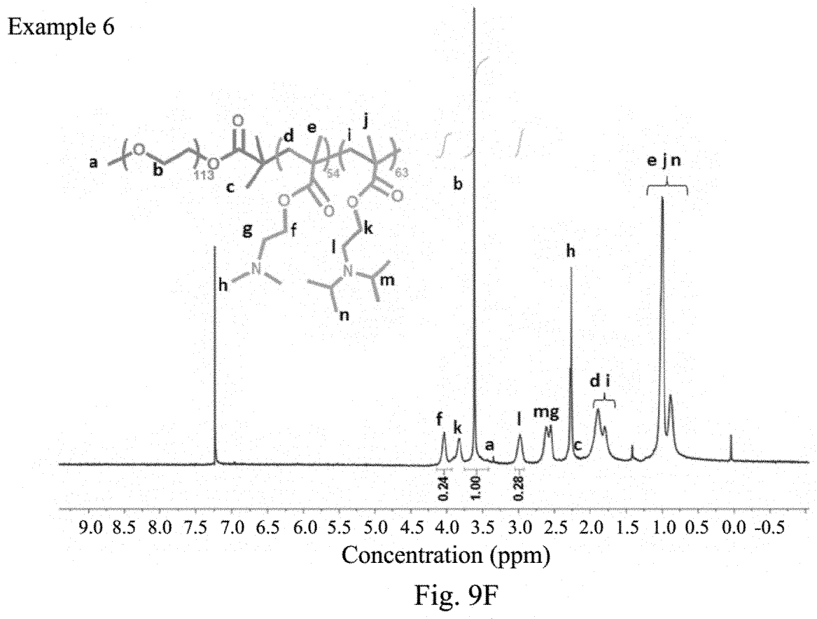

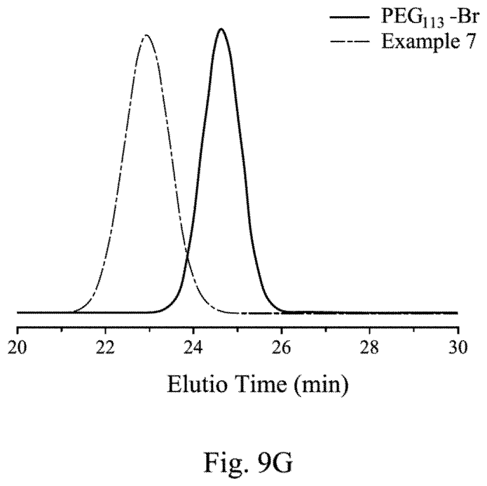

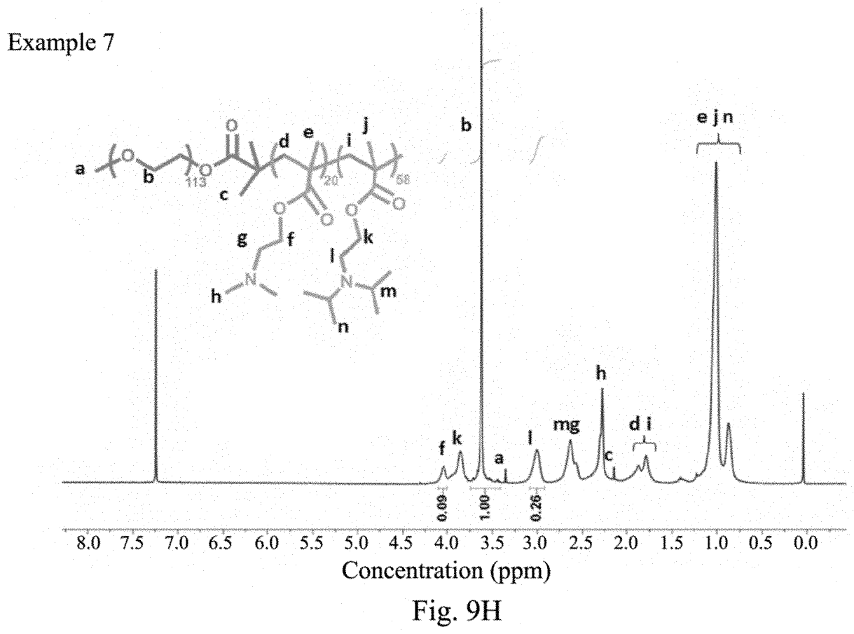

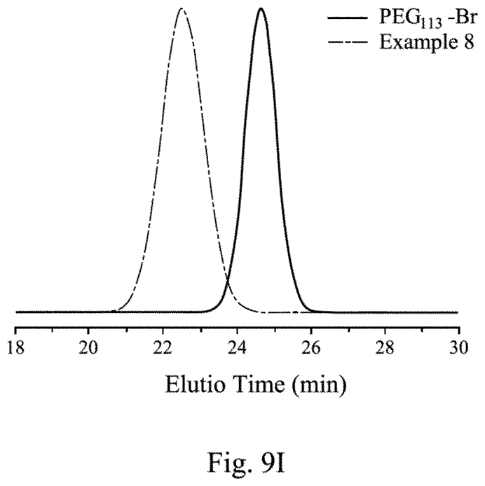

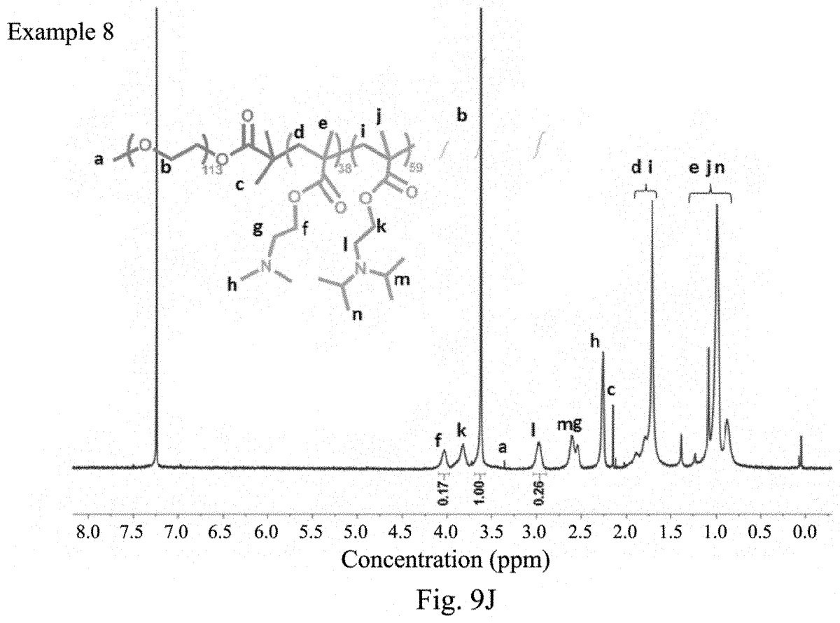

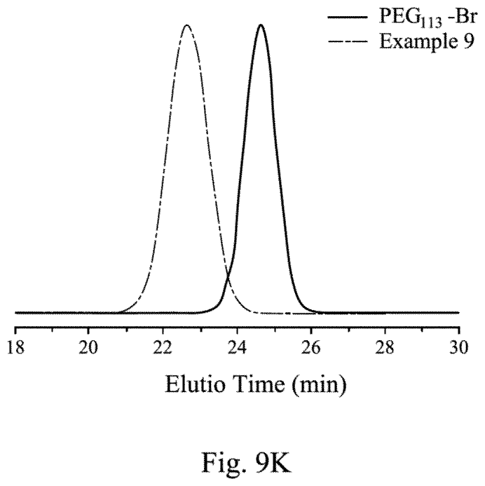

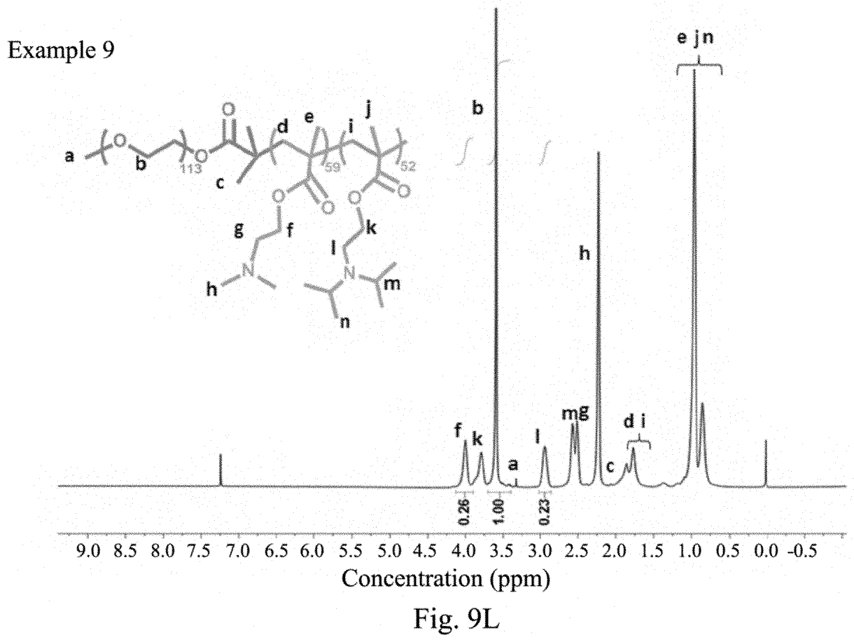

Please refer to FIGS. 9A, 9B, 9C, 9D, 9E, 9F, 9G, 9H, 9I, 9J, 9K and 9L and Table 2. FIG. 9A is a gel permeation chromatogram for the drug carrier of Example 4. FIG. 9B is a ¹H-NMR spectrum for the drug carrier of Example 4. FIG. 9C is a gel permeation chromatogram for the drug carrier of Example 5. FIG. 9D is a ¹H-NMR spectrum for the drug carrier of Example 5. FIG. 9E is a gel permeation chromatogram for the drug carrier of Example 6. FIG. 9F is a ¹H-NMR spectrum for the drug carrier of Example 6. FIG. 9G is a gel permeation chromatogram for the drug carrier of Example 7. FIG. 9H is a ¹H-NMR spectrum for the drug carrier of Example 7. FIG. 9I is a gel permeation chromatogram for the drug carrier of Example 8. FIG. 9J is a ¹H-NMR spectrum for the drug carrier of Example 8. FIG. 9K is a gel permeation chromatogram for the drug carrier of Example 9. FIG. 9L is a ¹H-NMR spectrum for the drug carrier of Example 9.

After confirmation by nuclear magnetic resonance, the repeating unit number of each segment of the drug carrier of Example 4 is PEG₁₁₃-b-PDMAEMA₁₆-b-PDPA₄₈, the repeating unit number of each segment of the drug carrier of Example 5 is PEG₁₁₃-b-PDMAEMA₂₀-r-PDPA₅₈, the repeating unit number of each segment of the drug carrier of Example 6 is PEG₁₁₃-b-PDMAEMA₄₃-b-PDPA₅₄, the repeating unit number of each segment of the drug carrier of Example 7 is PEG₁₁₃-b-PDMAEMA₃₈-r-PDPA₅₉, the repeating unit number of each segment of the drug carrier of Example 8 is PEG₁₁₃-b-PDMAEMA₅₄-b-PDPA₆₃, and the repeating unit number of each segment of the drug carrier of Example 9 is PEG₁₁₃-b-PDMAEMA₅₉-r-PDPA₅₂. The analytical results of the number average molecular weight (M_{n,NMR}), the polydispersity index (PDI) and the degree of polymerization (DP) of the drug carrier of Examples 4 to 9 are shown in Table 2.

TABLE 2

| | | | DP | |
|---|---|---|---|---|
| Drug carrier | M_{n, NMR} (g mol⁻¹) | PDI | DMAEMA | DPA |
| Example 4 | 17,700 | 1.17 | 16 | 48 |
| Example 5 | 20,500 | 1.16 | 20 | 58 |
| Example 6 | 23,200 | 1.27 | 43 | 54 |
| Example 7 | 23,500 | 1.22 | 38 | 59 |
| Example 8 | 26,900 | 1.25 | 54 | 63 |
| Example 9 | 25,300 | 1.18 | 59 | 52 |

As shown in Table 2, the polydispersity index of the drug carrier of Example 4, the drug carrier of Example 5, the drug carrier of Example 6, the drug carrier of Example 7, the drug carrier of Example 8, and the drug carrier of Example 9 of the present disclosure ranges from 1.16 to 1.27, wherein the PDI value is closer to 1, the molecular weight of the polymer is more uniform. The results indicate that the molecular weight of the drug carrier of PEGₙ-PDMAEMAₘ-PDPA_{b} of the 2nd embodiment is distributed uniformly. The drug carrier of Example 4, the drug carrier of Example 6, and the drug carrier of Example 8 is the triblock copolymer, respectively. The drug carrier of Example 5, the drug carrier of Example 7, and the drug carrier of Example 9 is the random copolymer, respectively.

### 2.2. Drug Loading Efficiency and Release Analysis of Active Substance of a Drug Carrier of the 2nd Embodiment

The composition of the drug carrier of the 2nd embodiment of the present disclosure has been fully described through the foregoing material analysis.

Subsequently, the drug carrier of Examples 4 to 9 is dissolved in tetrahydrofuran (THF)/dimethylformamide (DMF) mixed solvent with a hydrophobic drug, respectively. The hydrophobic drug is encapsulated in the drug carrier of Example 4, the drug carrier of Example 5, the drug carrier of Example 6, the drug carrier of Example 7, the drug carrier of Example 8, and Example 9 under sonication to form the drug delivery system of Example 4, the drug delivery system of Example 5, the drug delivery system of Example 6, the drug delivery system of Example 7, the drug delivery system of Example 8, and the drug delivery system of Example 9. The drug delivery system of Examples 4 to 9 are dialyzed and washed with phosphate buffered saline (PBS) and then lyophilized. After dissolving the drug delivery system of Examples 4 to 9 in the THF/DMF mixed solvent, the particle size before and after encapsulating the drug of the drug delivery system of Examples 4 to 9 is detected using the dynamic light scattering (DLS), and the absorbance at the maximum excitation wavelength (λex) 480 nm of the drug delivery system of Examples 4 to 9 is detected to determine the drug loading efficiency (DLE) and drug loading content (DLC). The hydrophobic drug encapsulated in the drug delivery system of the 2nd embodiment is doxorubicin (DOX) in this experiment, but the hydrophobic drug encapsulated in the drug delivery system of the present disclosure is not limited thereto.

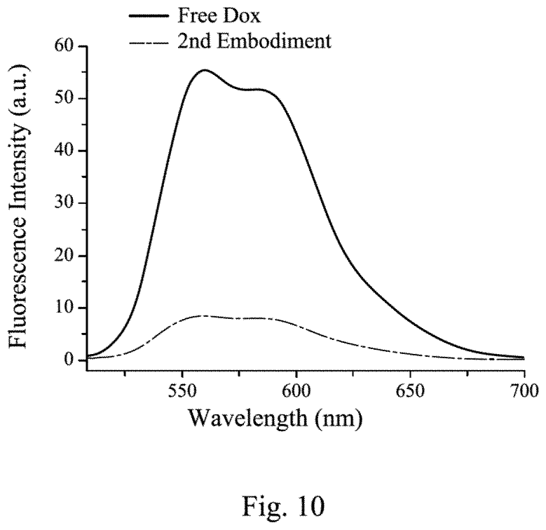

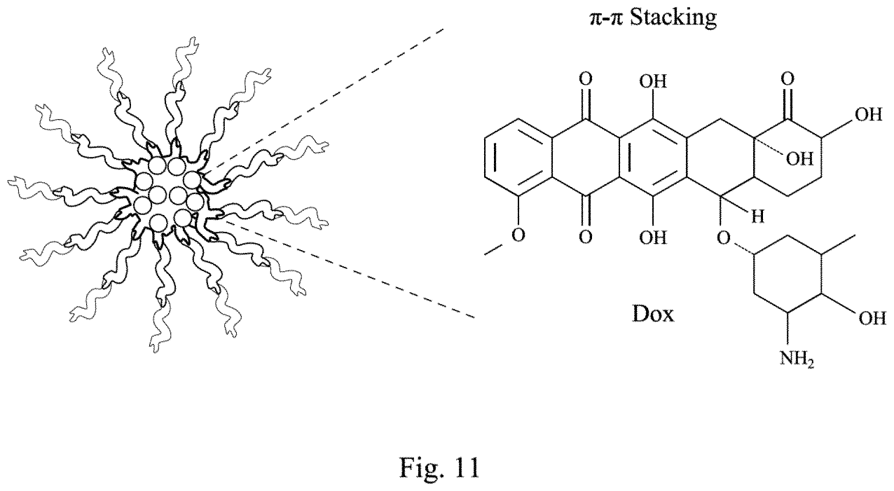

Please refer to FIGS. 10 and 11 and Table 3. FIG. 10 shows analytical result of drug loading efficiency of the drug delivery system according to the 2nd embodiment of the present disclosure. FIG. 11 is a schematic view of an active substance encapsulated in the drug delivery system according to the 2nd embodiment of the present disclosure. Table 3 shows the analytical results of the particle size before and after encapsulating the hydrophobic drug, the drug loading efficiency (DLE) and the drug loading content (DLC) of the drug delivery system of Examples 4 to 9.

TABLE 3

| drug delivery | Size (nm) before | Size (nm) after | | |
|---|---|---|---|---|
| system | encapsulating | encapsulating | DLE (%) | DLC (%) |
| Example 4 | 46.6 ± 1.0 | 79.4 ± 7.6 | 70,6 ± 3.3 | 9.6 ± 0.4 |
| Example 5 | 50.9 ± 1.5 | 64.6 ± 7.4 | 59.6 ± 7.6 | 8.2 ± 1.0 |
| Example 6 | 49.9 ± 2.3 | 66.6 ± 4.6 | 69.7 ± 5.5 | 9.5 ± 0.7 |
| Example 7 | 48.3 ± 1.0 | 90.2 ± 10.7 | 61.0 ± 7.3 | 8.4 ± 0.9 |
| Example 8 | 58.5 ± 1.6 | 68.2 ± 4.1 | 68.9 ± 3.7 | 9.4 ± 0.5 |
| Example 9 | 46.6 ± 1.0 | 150.7 ± 12.3 | 61.5 ± 1.7 | 8.4 ± 0.2 |

As shown in Table 3, the particle sizes of the drug delivery system of Examples 4 to 9 are all increased after encapsulating with doxorubicin. In the drug delivery system of the 2nd embodiment using the triblock copolymer as the drug carrier, the drug loading efficiency is about 70% (70.6%±3.3% in Example 4, 69.7%±5.5% in Example 6, and 68.9%±3.7% in Example 8), and the drug loading content is about 10% (9.6%±0.4% in Example 4, 9.5%±0.7% in Example 6, and 9.4%±0.5% in Example 8). The results indicate that the drug delivery system of the 2nd embodiment has a good loading effect and doxorubicin accounts for 10% by weight of the entire drug delivery system of the 2nd embodiment. In the drug delivery system of the 2nd embodiment using the random copolymer as the drug carrier, the drug loading efficiency is about 60% (59.6%±7.6% in Example 5, 61.0%±7.3% in Example 7, and 61.5%±1.7% in Example 9), and the drug loading content is about 8% (8.2%±1.0% in Example 5, 8.4%±0.9% in Example 7, and 8.4%±0.2% in Example 9).

In order to verify that doxorubicin is successfully encapsulated in the hydrophobic core of the drug delivery system of the 2nd embodiment, the fluorescence intensity at wavelengths from 500 nm to 700 nm of the drug delivery system of the 2nd embodiment encapsulated with doxorubicin and the same concentration of free doxorubicin is detected, respectively. In FIGS. 10 and 11, the fluorescence is quenched when doxorubicin is encapsulated in the drug delivery system of the 2nd embodiment, which is a self-quenching caused by the π-π stacking effect of doxorubicin in the hydrophobic core of the drug delivery system of the 2nd embodiment. Therefore, it can be proved that the doxorubicin is successfully encapsulated in the drug delivery system of the 2nd embodiment, and the particle size of the drug delivery system of the 2nd embodiment is increased after encapsulating the doxorubicin, which mainly caused by an expansion causing by doxorubicin of the hydrophobic core of the drug delivery system of the 2nd embodiment.

The drug carrier of the 2nd embodiment is expected to be responsive to the change in pH value to release the hydrophobic drug encapsulated in the drug delivery system because the drug carrier of the 2nd embodiment contains a pH-responsive cleavage segment. The drug delivery system of Example 6 is dissolved in phosphate buffered saline at pH 7.4, 6.0, and 5.0, respectively, and the release rate of the pharmaceutically active substance of the drug delivery system of Example 6 at different pH values is analyzed.

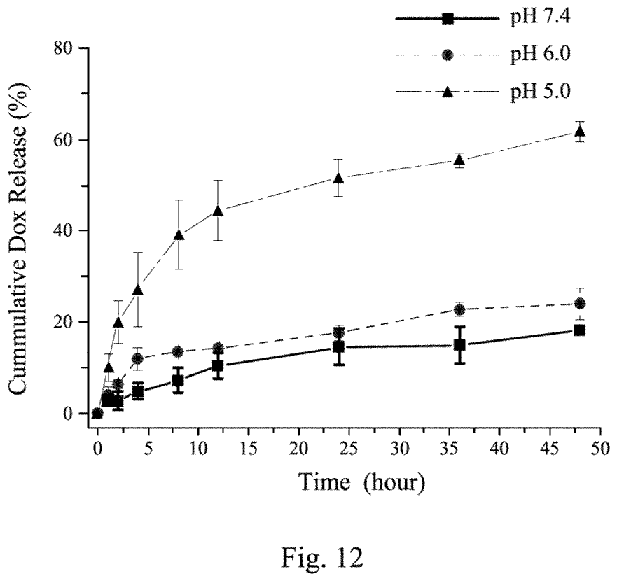

Please refer to FIG. 12, which shows the analytical result of cumulative active substance release of a drug delivery system of Example 6. In FIG. 12, at pH 7.4 and 6.0, the cumulative doxorubicin release rate of the drug delivery system of Example 6 is only 10% within 5 hours and only nearly 20% within 50 hours. However, at pH 5.0, the cumulative doxorubicin release rate of the drug delivery system of Example 6 is close to 30% within 5 hours and further closer to 60% within 50 hours. The results indicate that the negatively charged carboxyl group formed by the PDPA segment helps release the drug delivery system encapsulated in the 2nd embodiment by lowering the pH value.

To sum up, the drug carrier of the present disclosure can efficiently encapsulate the nucleic acid or the active substance to form the drug delivery system. The drug delivery system of the present disclosure has good biocompatibility, is nontoxic to cells, has high storage stability, and can selectively release the nucleic acid or the active substance encapsulated therein by a light modulation or an acid modulation. Therefore, the drug delivery system of the present disclosure can be used in gene therapy and drug therapy to solve the problem of low drug responsiveness in clinical trials.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A drug carrier comprising a structure represented by formula (1): 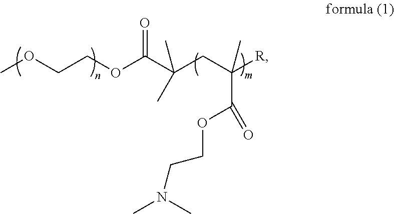
wherein n is a number greater than 30 and less than 150, m is a number greater than 30 and less than 120, R is a structure represented by formula (2) or formula (3): 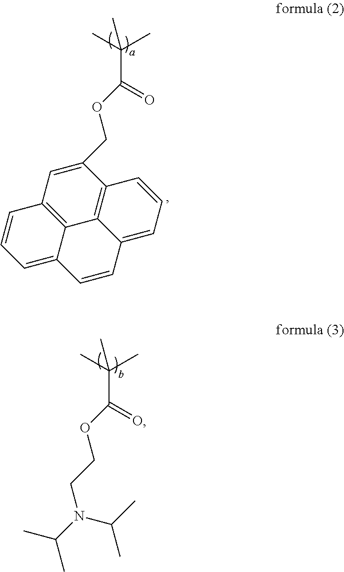
wherein a is a number greater than 30 and less than 100, b is a number greater than 30 and less than 100.

2. The drug carrier of claim 1, wherein a polydispersity index of the drug carrier is 1.1 to 2.0.

3. A drug delivery system comprising the drug carder of claim 1 and an effective amount of a nucleic acid, wherein the nucleic acid is encapsulated in the drug carrier.

4. The drug delivery system of claim 3, wherein the nucleic acid is selected from the group consisting of an oligo-double-stranded DNA, a poly-double-stranded DNA, an oligo-single-stranded DNA, a poly-single-stranded DNA, an oligo-single-stranded RNA and a poly-single-stranded RNA.

5. The drug delivery system of claim 4, wherein the nucleic acid is a plasmid DNA, a small interfering RNA (siRNA), a microRNA (miRNA), an antisense RNA (asRNA), a decoy nucleic acid or an aptamer.

6. The drug delivery system of claim 3, wherein a ratio of a concentration of amine groups in the drug carrier to a concentration of phosphate groups in the nucleic acid is an N/P ratio, and the N/P ratio is higher than or equal to 5.

7. The drug delivery system of claim 3, wherein the nucleic acid is released by a photo irradiation.

8. The drug delivery system of claim 3, wherein the nucleic acid is released by lowering a pH value to a release pH value, and the release pH value is less than or equal to 5.

9. A drug delivery system comprising the drug carrier of claim 1 and an effective amount of an active substance, wherein the active substance is encapsulated in the drug carrier.

10. The drug delivery system of claim 9, wherein the active substance is a hydrophobic drug.

11. The drug delivery system of claim 10, wherein the hydrophobic drug is doxorubicin (DOX), tamoxifen, irinotecan, paclitaxel or sorafenib.

12. The drug delivery system of claim 9, wherein a loading concentration of the active substance in the drug carrier is greater than 0 mg/mL and less than 100 mg/mL.

13. The drug delivery system of claim 9, wherein the active substance is released by a photo irradiation.

14. The drug delivery system of claim 9, wherein the active substance is released by lowering a pH value to a release pH value, and the release pH value is less than or equal to 5.
